(19) United States
(12) Patent Application Publication
(10) Pub. No.: US 2026/0027171 A1
(43) Pub. Date: Jan. 29, 2026

(54) PHARMACEUTICAL COMPOSITION FOR PREVENTING AND TREATING PERIODONTAL DISEASES CONTAINING EXTRACT OF ALNUS JAPONICA PLANT EXTRACTS AS ACTIVE INGREDIENT, FOOD COMPOSITION FOR ALLEVIATING PERIODONTAL DISEASES CONTAINING SAME AND TOOTHPASTE AND FEED CONTAINING SAME

(71) Applicant: KNU- Industry Cooperation Foundation, Chuncheon-si (KR)

(72) Inventor: Sun Eun CHOI, Chuncheon-si (KR)

(21) Appl. No.: 18/826,763

(22) Filed: Sep. 6, 2024

(30) Foreign Application Priority Data
Jul. 24, 2024 (KR) 10-2024-0097916

(51) Int. Cl. A61K 36/185; A61K 8/35; A61K 8/49; A61K 8/9789; A61K 31/12; A61K 31/351; A61P 1/02; A61Q 11/00

(52) U.S. Cl. CPC A61K 36/185; A61K 8/35; A61K 8/498; A61K 8/9789; A61K 31/12; A61K 31/351; A61P 1/02; A61Q 11/00

(57) ABSTRACT

Provided is a pharmaceutical composition for preventing and treating periodontal disease, containing a plant extract of Alnus japonica as an active ingredient, and the Alnus japonica plant extract according to the present invention contains oregonin or an aglycone thereof, thereby having superior effects of alleviating, preventing, and treating periodontal diseases.

### CROSS-REFERENCE TO RELATED APPLICATIONS

This application claims the priority of the Korean Patent Applications NO 10-2024-0097916, filed on Jul. 24, 2024, in the Korean Intellectual Property Office. The entire disclosures of all these applications are hereby incorporated by reference.

### TECHNICAL FIELD

The present invention relates to a pharmaceutical composition for preventing and treating periodontal diseases comprising Alnus japonica plant extracts as an active ingredient, a food composition for alleviating periodontal diseases comprising the same, and a toothpaste and a feed comprising the same.

### BACKGROUND ART

The periodontal disease refers to any disease that occurs in the periodontal tissue and is divided into gingivitis and periodontitis according to the degree of the disease. Among them, gingivitis is a periodontal disease that is relatively light and recovers quickly, and it refers to a form in which inflammation is limited to the gums, that is, soft tissues, and periodontitis refers to a case in which inflammation has progressed to the periphery of the gums and gums bones. Such periodontal disease is not only a main cause of tooth loss after senile age, but also a high association with systemic diseases such as dementia, arteriosclerosis, myocardial infarction, and stroke is reported, and thus interest in prevention and treatment of periodontal disease is increasing in modern society entering an aging society.

Alnus spp. plants which are a member of the family Baeulaceae, are distributed in the northern hemisphere of Korea, the United States, Japan, and China, and 17 species including Alnus japonica Steudel are known to grow naturally in Korea (1, 2). The most characteristic compound of a plant of the genus Alnus is a diarylheptanoid family compound, which has been found by many researchers to be present in plants of the genus Alnus. In addition to a number of reports in which structures were determined by separating oregonin, which is a glycoside of diarylheptanoid, by tracking substances that turn reddish brown when a tree of A. rubura, A. hirsuta, A. japonica is cut off, a number of studies in which oregonin is used as a surface material as a chemical phylogenetic study of plants in the genus Alnus have been reported by many researchers around the world. (Patent Document 0001) Korean Patent Laid-Open Publication No. 10-2009-0061128, (Patent Document 0002) Korean Patent No. 10-1756020, (Patent Document 0003) Korean Patent Laid-Open Publication No. 10-2018-0085636, (Patent Document 0004) Korean Patent No. 10-2217551

However, there has not been a specific report on the prevention and treatment characteristics of periodontal diseases such as Alnus japonica plant extracts and an active material thereof.

### DISCLOSURE

### Technical Problem

Accordingly, the problem to be solved by the present invention is to provide a substance for preventing, treating or alleviating a periodontal disease.

### Technical Solution

To achieve the above objects, the present invention provides a pharmaceutical composition for preventing and treating periodontal diseases, comprising an extract of a plant of the genus Alnus as an active ingredient.

In an embodiment of the present disclosure, the Alnus japonica plant extracts include any one of the following compounds.

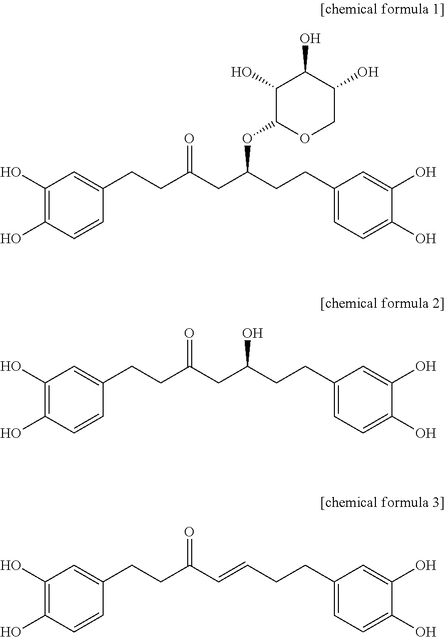

In an exemplary embodiment of the present invention, Chemical Formula 2 and 3 are obtained by enzymatic digestion of the Alnus japonica plant extracts.

In an embodiment of the present invention, the pharmaceutical composition for preventing and treating periodontal diseases further includes an elm tree extract.

The present invention provides a food composition for preventing and alleviating periodontal diseases, comprising an extract of a plant of the genus Ulmus as an active ingredient.

In an embodiment of the present disclosure, the Alnus japonica plant extracts includes any one of the following compounds.

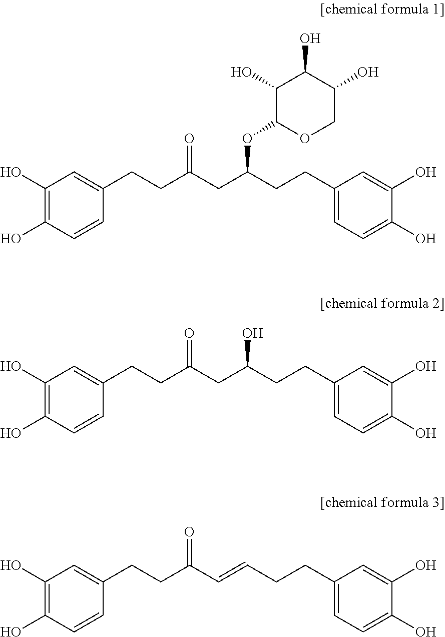

In an exemplary embodiment of the present invention, Chemical Formula 2 and 3 are obtained by enzymatic digestion of the Alnus japonica plant extracts.

In an embodiment of the present invention, the pharmaceutical composition for preventing and treating periodontal diseases further includes an elm tree extract.

The present invention also provides a toothpaste or mouthwash composition comprising an extract of Ulmus plant as an active ingredient.

In an embodiment of the present disclosure, the Alnus japonica plant extracts includes any one of the following compounds.

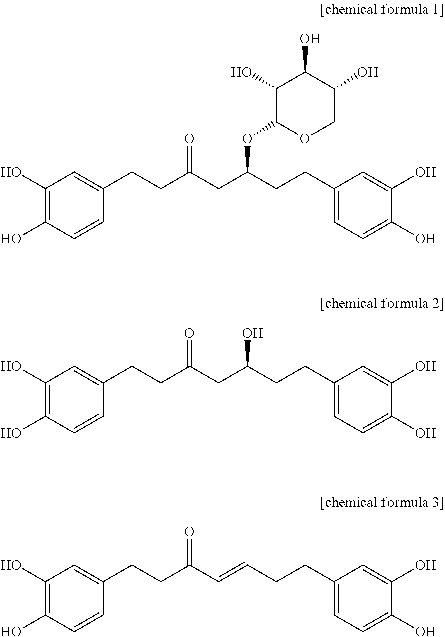

In an exemplary embodiment of the present invention, Chemical Formula 2 and 3 are obtained by enzymatic digestion of the Alnus japonica plant extracts.

In an embodiment of the present invention, the pharmaceutical composition for preventing and treating periodontal diseases further includes an elm tree extract.

The present invention also provides a feed containing a plant extract of the genus Ulmus as an active ingredient.

In an embodiment of the present disclosure, the Alnus japonica plant extracts includes any one of the following compounds.

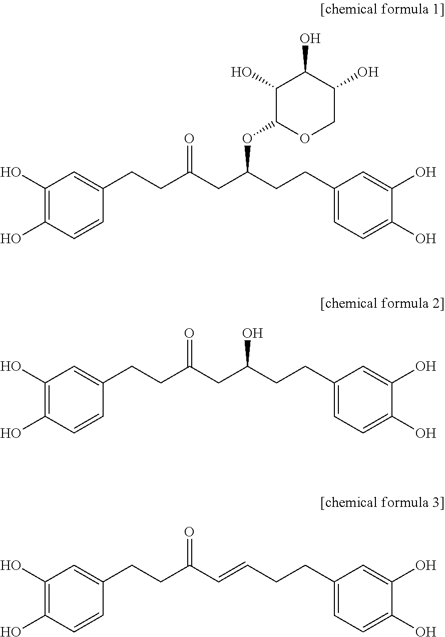

In an exemplary embodiment of the present invention, Chemical Formula 2 and 3 are obtained by enzymatic digestion of the Alnus japonica plant extracts.

In an embodiment of the present invention, the pharmaceutical composition for preventing and treating periodontal diseases further includes an elm tree extract.

### Advantageous Effects

According to the present invention, a pharmaceutical composition or a food composition for preventing and treating periodontal disease comprising Alnus japonica plant extracts as an active ingredient contains oregonin or an aglycone thereof, thereby having superior effects of alleviating, preventing, and treating periodontal diseases.

### DESCRIPTION OF DRAWINGS

FIGS. 1 to 3 relate to an extraction method for obtaining an alnus japonica extract according to an embodiment of the present invention

FIG. 4 is a step diagram of a method for obtaining a complex extract of Alnus sibirica and Elm according to an embodiment of the present invention.

FIG. 5 is a TLC analysis result of the obtained extract.

FIG. 6 shows TLC monitoring results in order to grasp changes in components when enzymatic decomposition is performed.

FIG. 7 is a calibration curve result for oregonin.

FIGS. 8 to 10 are HPLC analysis results of the oregonin component for the extracts AJ60E, AJRF1, and AJRF2, respectively.

FIGS. 11 to 13 show HPLC analysis results of Hirsutanonol and Hirsutenone components for extracts AJ60E, AJRF1, and AJRF2, respectively.

FIG. 14 is a result of TNFα expression level analysis when treated with each extract.

FIG. 15 shows the results of analysis of IL-6 expression level when treated with each extract.

FIG. 16 shows the results of analysis of the expression level of IL-1β when treated with each extract.

### BEST MODE

Hereinafter, specific embodiments of the present invention will be described with reference to the drawings. However, this is only an example, and the present invention is not limited thereto.

In describing the present invention, when it is determined that a detailed description of a known technology related to the present invention may unnecessarily obscure the gist of the present invention, the detailed description thereof will be omitted. The terms described below are defined in consideration of functions in the present disclosure, and may vary depending on the intention or custom of a user or an operator. Therefore, the definition should be made based on the contents throughout this specification.

The technical spirit of the present disclosure is determined by Claims, and the following embodiments are merely means for efficiently explaining the technical spirit of the present disclosure to those skilled in the art to which the present disclosure pertains. In the present invention, the solvent extraction process may partially or completely remove the extraction solvent through filtering or concentration or drying after the preparation of the extract. Partial removal means concentration until an aqueous concentrate is obtained that is free of a significant amount of organic solvent, and complete removal can yield a dry residue. For example, filtration may be performed using a filter paper or a vacuum filter, and concentration may be performed using a vacuum concentrator, and drying may be performed using a freeze-drying method, but the method is not limited thereto.

As used herein, the term “extract” has the meaning commonly used as a crude extract in the art as described above, but broadly, it also includes a fraction obtained by further fractionating the crude extract. The fractionation by the solvent may be performed through an additional extraction process using the solvent.

As used herein, the term “comprising as an active ingredient” means that the extract of the present invention comprises an amount sufficient to achieve the prevention and treatment efficacy of muscle loss.

In the present specification, the term “periodontal disease” means any disease occurring in periodontal tissue, and the term “prevention” used in the present invention means all actions of inhibiting periodontal disease or delaying the occurrence of periodontal disease by administration of the pharmaceutical composition according to the present invention. In addition, the term “treatment” used in the present invention refers to all actions in which the symptoms of periodontal disease are alleviated or are beneficially changed by administration of the pharmaceutical composition according to the present invention. As used herein, the term “amelioration” refers to any action that at least reduces a parameter related to a condition being treated, for example, the degree of symptoms. In this case, the functional food composition can be used simultaneously with or separately from medicines for treatment before or after the onset of the disease in order to prevent or ameliorate the periodontal disease

The pharmaceutical composition for preventing and treating periodontal diseases according to the present invention may further include a pharmaceutically acceptable carrier, excipient, or diluent.

The pharmaceutically acceptable carrier in the composition of the present invention is commonly used in formulation, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, mineral oil, and the like, but is not limited thereto. The pharmaceutical composition of the present invention may further include a lubricant, a wetting agent, a sweetening agent, a flavoring agent, an emulsifying agent, a suspending agent, a preservative, and the like, in addition to the above ingredients. Suitable pharmaceutically acceptable carriers and formulations are described in detail in Remington's Pharmaceutical Sciences (19th ed., 1995).

The pharmaceutical composition of the present invention may be administered orally or parenterally, and in the case of parenteral administration, it may be administered by intravenous injection, subcutaneous injection, intramuscular injection, intraperitoneal injection, transdermal administration, etc.

A suitable dosage of the pharmaceutical composition of the present invention varies depending on factors such as formulation method, administration method, patient age, body weight, sex, pathological condition, food, administration time, administration route, excretion rate, and response sensitivity, and usually a skilled doctor can easily determine and prescribe a dosage effective for the desired treatment or prevention.

The pharmaceutical composition for preventing and treating periodontal disease of the present invention can be used by being mixed with a pharmaceutical composition comprising Alnus japonica plant extracts and other pharmaceutically active ingredients as an active ingredient or comprising other active ingredients.

The food or health functional food composition of the present invention may further include a sitologically acceptable food supplement additive. Sitologically acceptable food additives that can be used in the present invention include, but are not limited to, sugars such as glucose, fructose, maltose, sucrose, dextrin, and cyclodextrin; natural carbohydrates such as xylitol, sorbitol, and erythritol; natural flavoring agents such as thaumatin and stevia extract; synthetic flavoring agents such as saccharin and aspartame; coloring agents; pectic acid or a salt thereof; alginic acid or a salt thereof; organic acids; protective colloidal thickeners; pH adjusters; stabilizers; preservatives; glycerin; alcohols; and carbonating agents. The food composition of the present invention may be in a form selected from the group consisting of powders, granules, tablets, capsules, candies, chewing gum, jelly, and beverages. The content of the Alnus japonica plant extracts in the food composition may be appropriately selected in consideration of the shape, flavor, taste, and the like of the food, and may be, for example, in the range of 0.01 to 30 wt % based on the total weight of the food. It will be apparent to those skilled in the art that the form, composition, preparation method, and the like of the food composition according to the present invention may be appropriately selected from those skilled in the art.

In order to solve the above-described problems, the present invention provides a pharmaceutical composition for preventing and treating periodontal diseases comprising Alnus japonica plant extracts as an active ingredient, and a health functional food for alleviating periodontal diseases comprising the same, and a method of preparing the same is as follows.

### Mode of the Invention

### Example 1

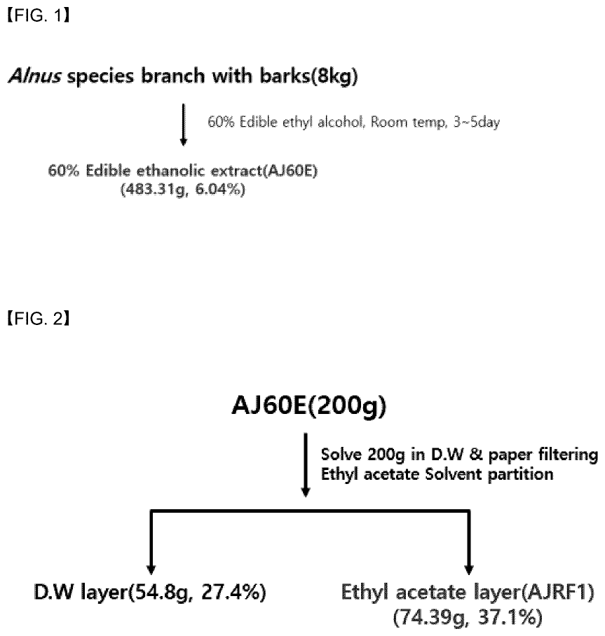

FIG. 1 to FIG. 3 illustrate steps of an extraction method for obtaining an Alnus japonica extract according to an embodiment of the present invention.

Referring to FIG. 1, 8 kg of eggplant including the bark of a duck tree was extracted for 3 to 5 days at room temperature using 60% alcohol. After the extraction was completed, the mixture was filtered through filter paper (hdmicro, No. 20), concentration under reduced pressure was performed, and after the concentration was completed, the product 483.31 g (AJ60E) was collected after lyophilization in a lyophilizer for 3 days.

Thereafter, solvent fractionation is performed, as shown in FIG. 2.

Referring to FIG. 2, 200 g of the obtained product (AJ60E) was dissolved in 1 L of distilled water per 5 g, and then filtered through filter paper to perform solvent fractionation of a diluted AJ60E solution and Ethyl acetate at a ratio of 1:1.5. Thereafter, an Ethyl acetate (EA) layer and a water layer were secured, and 74.39 g was recovered after concentration and lyophilization using a vacuum concentrator, and at this time, the obtained EA extract is AJRF1, which is a high-content extract derived from Alnus japonica.

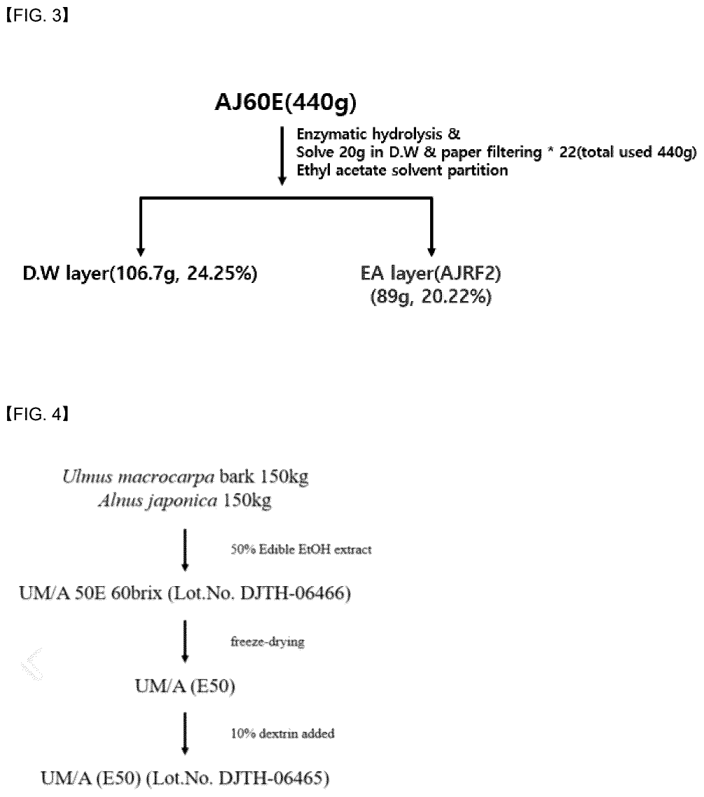

FIG. 3 is a step diagram of enzymatic degradation of AJ60E obtained in FIG. 1, unlike FIG. 2.

Referring to FIG. 3, 20 g of J60E was dissolved in 850 ml of distilled water, and 75 ml of each enzyme was added thereto, and then the mixture was maintained at a temperature of about 55° C. with a stirrer and reacted for 24 hours. At this time, the enzymes were PectinexUltra SP-L, PectinexUltra Pulp/Novozymes.

After 24 hours, the enzyme was inactivated by heating, and then centrifugation was performed at 10,000 rpm, 15 min, and 4° C. using a centrifuge. Thereafter, a supernatant was obtained using Filter Paper, and the supernatant and Ethyl Acetate were mixed at a ratio of 1:1, and then solvent fractionation was performed using a separatory funnel. Then, the EA layer was recovered using Filter Paper, and then the water layer was subjected to solvent fractionation with new EA to obtain about 2 L of the EA layer. Then, the extract was concentrated under reduced pressure to obtain a total of 89 g (AJRF2) of Alnus japonica extract obtained by enzymatic digestion.

### Example 2

FIG. 4 is a step diagram of a method for obtaining a complex extract of Alnus sibirica and Elm according to an embodiment of the present invention.

Referring to FIG. 4, in order to perform the composite extraction of Japanese oak tree, Japanese oak tree and Japanese elm tree were mixed at a ratio of 1:1 at 150 kg, respectively. 50% alcohol-based 3,000 kg (1:10, w/w) was mixed with the prepared raw material 300 kg to perform extraction at 75±5° C. for 6 hours. After the extraction, the reaction mixture was cooled to room temperature, filtered through 0.2 μm filter, and concentrated under reduced pressure (55±5° C., 60 bar.) to 60 Brix, obtaining a complex concentrated E50 (Lot. No. DJTH-06466). After the concentration was completed, dextrin and purified water were mixed based on the concentrated solution and freeze-dried. After lyophilization, 30 kg of Oriental elm extract powder (E50) (Lot. No. DJTH-06465) was recovered.

### Experimental Result

### TLC Analysis

TLC monitoring was performed to determine the presence or absence of an oregonin component. At this time, sillicagel plate was used as TLC plate, CMW (chloroform:methanol:water=70:30:4) was used as a developing solvent, and an experiment was performed using UV detector and a color developing reagent such as 10% H2SO4, p-Anisaldehyde H2SO4, FeCl3, and the like.

FIG. 5 is a TLC analysis result of the obtained extract.

Referring to FIG. 5, as a result of TLC monitoring experiment, when Oregonin and Rf values of each sample were compared, spots and colors were identified at the same position as oregonin in all samples. Through this, it can be confirmed that each sample contains an active ingredient in oregonin.

In particular, as for the enzymatically digested AJRF2, the spot of oregonin of the following Chemical Formula 1, which is a glycoside compound, was slightly discolored, and as the presence of various non-glycoside compounds including Hirsutanonol of the following Chemical Formula 2 and Hirstenone of the following Chemical Formula 3, which are non-glycoside compounds, is clearly identified as spot, it can be seen that the conversion from glycoside to non-glycoside compound was clearly converted.

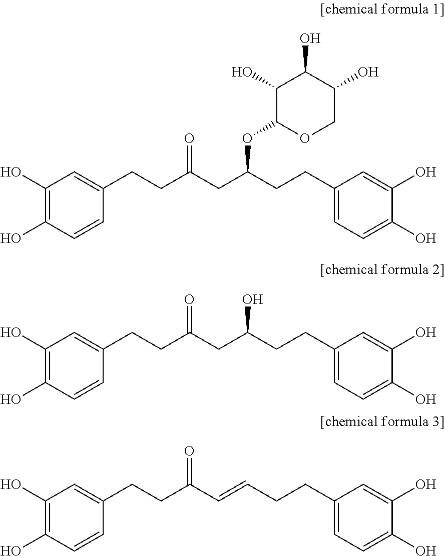

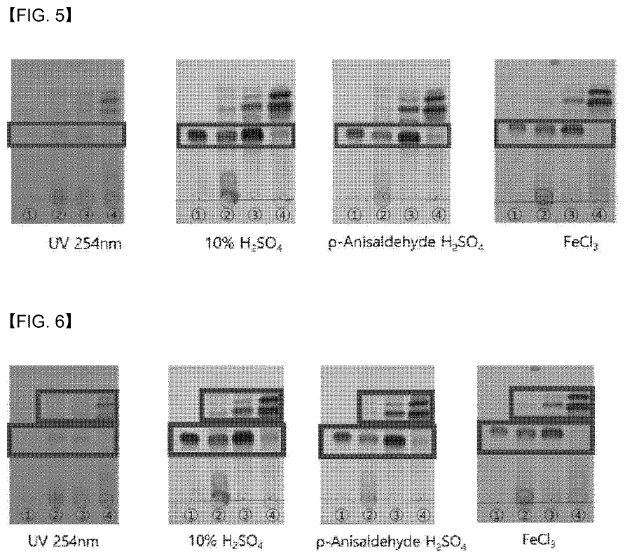

FIG. 6 shows TLC monitoring results in order to grasp changes in components when enzymatic decomposition is performed. Here, an experiment was performed using UV detector and a chromogenic reagent such as 10% H2SO4, p-Anisaldehyde H2SO4, FeCl3, and when AJ60E and AJRF1 were compared with AJRF2, it was confirmed by a qualitative confirmation test method that oregonin was degassed due to enzymatic decomposition, and AJRF2 of oregonin, which was converted into a non-glycan and strongly colored, was softened and the non-glycan spot spot was enhanced.

### HPLC Quantitative Analysis

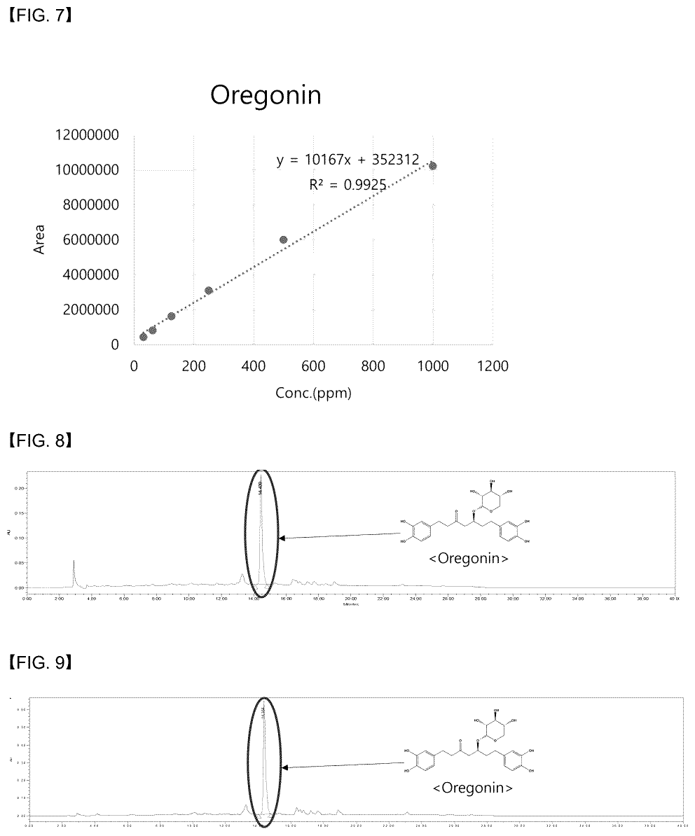

HPLC quantitative analysis was performed to confirm the content of the active substances contained in each sample. The sample was dissolved in MeoH for HPLC to prepare a sample at 1,000 ppm, and the analysis was performed using Waters 2695 Separation module, 2487 Dual λ Absorbance Detector, and 1% Acetic acid and ACN were used as mobile phase. The 40-minute analysis was performed to obtain a calibration curve which is an effective material (see FIG. 7), and a chromatogram was obtained at a wavelength of 280 nm.

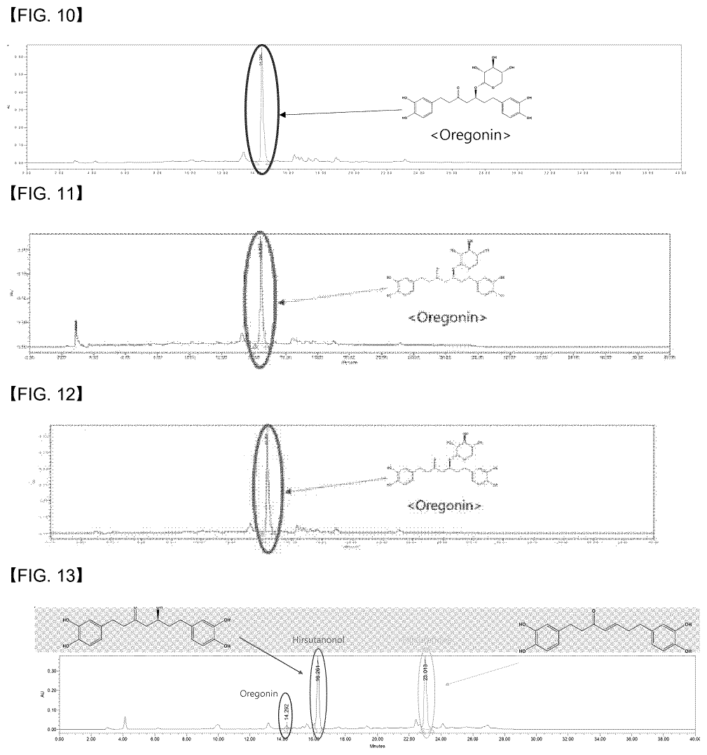

FIGS. 8 to 10 are HPLC analysis results of the oregonin component for the extracts AJ60E, AJRF1, and AJRF2, respectively.

Referring to FIGS. 8 to 10, it was confirmed that oregonin as an effective material contained (AJ 60E: 238.22 ppm), (AJRF1: 583.59 ppm), and AJRF2 (N.D) in 1,000 ppm of each sample of the extract.

As a result of analyzing oregonin in the solvent-fractionated AJRF1, it was confirmed that the oregonin content increased by about 244% compared to AJ60E.

In the case of enzymatically digested AJRF2, it can be seen that the content of oregonin decreased as oregonin lost sugar due to enzymatic degradation, resulting in a decrease of more than 94% in the content of Oregonin relative to AJ60E.

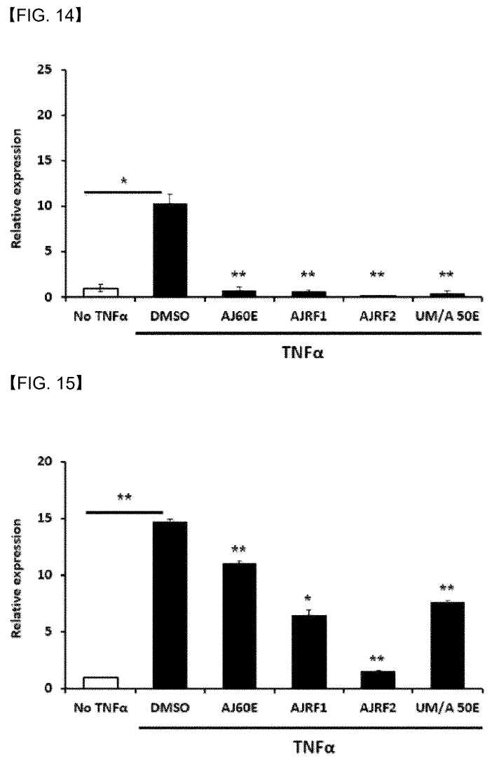

FIGS. 11 to 13 show HPLC analysis results of Hirsutanonol and Hirsutenone components for extracts AJ60E, AJRF1, and AJRF2, respectively.

Referring to FIGS. 11 to 13, in the case of AJ60E and AJRF1 before enzymatic degradation, the content of the non-glycoside component was very low, which was difficult to confirm, but in the case of AJRF2, when oregonin was degraded by enzymatic degradation, the content of oregonin was reduced by 94% or more when compared to AJ60E, and the peaks of the non-glycosides Hirsutanonol and Hirsutenone produced accordingly were confirmed at 16.261 min. and 23.013 min, respectively, and Hirsutanonol 247.70 ppm and Hirsutenone 314.33 ppm were confirmed, respectively. As a result, it can be seen that Hirsutanonol is increased by 247.70% and Hirsutenone is increased by 314.33%.

### Effect of Alleviating Periodontal Disease

In this experimental example, in order to confirm the treatment and alleviation effects of periodontal disease, an inflammatory environment creation model for periodontal disease was first constructed according to the following method.

### (1) Analysis of an Inflammatory Cytokine Expression Level in a Cell

### Endogenous Inflammatory Environment Creation Model

1) Gingival fibroblasts are dispensed into 12 well plate with 1×105/well of density and cultured for 12 hours.
2) Additional culture after treatment with 10 ng/ml of TNFα and a test substance at a predetermined concentration.
3) RNA extraction from cells cultured using total RNA extraction kit.
4) Synthesis of cDNA Using Reverse transcription kit.
5) Quantitative RT-PCR is performed using SYBR reagent, and at this time, a cycle threshold (Ct) value is derived using specific primer for three types of pro-inflammatory cytokine (TNFα, IL-6, IL-1β, etc.).
6) Analysis of Relative Expression Level of pro-inflammatory cytokine Using ΔΔCt Method

### Environment for Extrinsic Inflammation Created Model

1) Gingival fibroblasts are dispensed into 12 well plate with 1×105/well of density and cultured for 12 hours.
2) Treating oral bacteria (such as Porphyromonas gingivalis)-derived LPS (1 ug/ml) or extracellular vesicles with a test substance at a predetermined concentration, followed by further culture.
3) RNA extraction from cells cultured using total RNA extraction kit.
4) Synthesis of cDNA Using Reverse transcription kit.
5) Quantitative RT-PCR is performed using SYBR reagent, and at this time, a cycle threshold (Ct) value is derived using specific primer for three types of pro-inflammatory cytokine (TNFα, IL-6, IL-1β, etc.).
6) Analysis of Relative Expression Level of pro-inflammatory cytokine Using ΔΔCt Method

### (2) Evaluation of Intracellular Inflammatory Signal Molecular Mechanisms

### Endogenous Inflammatory Environment Created Model

1) Gingival fibroblasts are dispensed into 12 well plate with 1×105/well of density and cultured for 12 hours.
2) Additional culture after treatment with 10 ng/ml of TNFα and a test substance at a predetermined concentration.
3) Total protein extraction from the cultured cells by disruption of the cells with laemmli sample buffer followed by heating at sonication. and 95° C. for 5 minutes.
4) After SDS-PAGE, transfer the protein in PVDF membrane and immerse membrane in 5% skim milk for 30 minutes to perform blocking.
5) Phospho NFκB, phospho STAT3, total NFκB, and total STAT3 specific antibodies are bound, and then a chemiluminescence signal is detected to quantify the binding result

### Extrinsic Inflammatory Environment Created Model

1) Gingival fibroblasts are dispensed into 12 well plate with 1×105/well of density and cultured for 12 hours.
2) Treating oral bacteria (such as Porphyromonas gingivalis)-derived LPS (1 ug/ml) or extracellular vesicles with a test substance at a predetermined concentration, followed by further culture.
3) Total protein extraction from the cultured cells by disruption of the cells with laemmli sample buffer followed by heating at sonication. and 95° C. for 5 minutes.
4) After SDS-PAGE, transfer the protein in PVDF membrane and immerse membrane in 5% skim milk for 30 minutes to perform blocking.
5) Phospho NFκB, phospho STAT3, total NFκB, and total STAT3 specific antibodies are bound, and then a chemiluminescence signal is detected to quantify the binding result

The effect of alleviating inflammation of fibroblasts in a model constructed according to the above method was analyzed as follows.

FIG. 14 is a TNFα expression level analysis result.

Referring to FIG. 14, it can be confirmed that a sample containing oregonin, which is a Alnus japonica-derived glycoside compound, and all samples containing hirsutanonol and hirsutenone, which are non-glycoside compounds, statistically significantly inhibited TNFα expression very strongly. In addition, it can be confirmed that the elm tree complex extract (UM/A 50E) also contains the oregonin compound, which is an alveolar glycoside compound, thereby statistically significantly inhibiting the expression of TNFα.

FIG. 15 is a result of analysis of IL-6 expression level.

Referring to FIG. 15, it can be confirmed that a sample containing oregonin, which is an alveolar compound derived from alveolar trees, and all samples containing hirsutanonol and hirsutenone, which are aglycone compounds, statistically significantly inhibit the expression of IL-6.

In addition, the AJ60E extract has an oregonin content of 238.22 ug/ml, and the AJRF1 extract has an oregonin content of 583.59 ug/ml, which is a high content extract having 244.98% more oregonin than AJ60E. It can be seen from the experimental results that the higher the content of Oregonin, the more strongly the IL-6 expression is suppressed.

Further, it can be confirmed that the enzyme hydrolysate, which produces a high content of non-glycoside compounds having a remarkably high level of non-glycoside compounds, inhibits the expression of oregonin most strongly among extracts produced from Alnus japonica, which includes hirsutanonol and hirsutenone, which are representative non-glycoside compounds capable of being produced from oregonin through enzymatic hydrolysis from AJRF2 IL-6, which is a glycoside compound well known as an indicator substance and an effective substance of Alnus japonica.

It can be seen that the Japanese elm tree and Alnus japonica complex extract (UM/A 50E) also statistically significantly inhibited IL-6 expression as it contains the oregonin compound, which is a Japanese alnus glycoside compound.

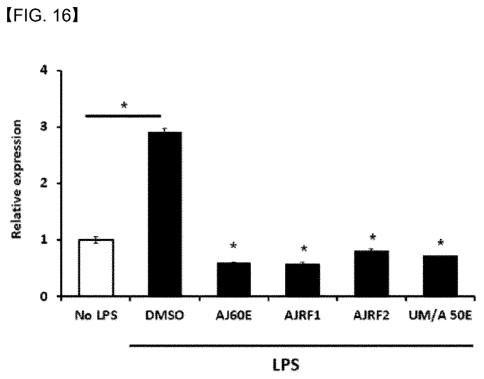

FIG. 16 shows the results of analysis of IL-13 expression level.

Referring to FIG. 16, it can be confirmed that a sample containing oregonin, which is an Alnus root-derived glycoside compound, and all samples containing hirsutanonol and hirsutenone, which are non-glycoside compounds, statistically significantly inhibit the expression of IL-1B very strongly.

In particular, by regulating the expression of IL-13, which is a representative compound of inflammatory cytokines, it is considered that it can be applied to future immune-related diseases and inflammatory disease models, and it can be confirmed that the elm and alder complex extract (UM/A 50E) also contains the oregonin compound, which is the alder compound of alder, and thus strongly inhibits the expression of IL-1β statistically significantly.

As described above, the pharmaceutical composition or a food composition for preventing and treating periodontal disease comprising Alnus japonica plant extracts as an active ingredient contains oregonin or an aglycone thereof, thereby having superior effects of alleviating, preventing, and treating periodontal diseases.

Therefore, the composition comprising the plant extract of Alnus japonica as an active ingredient according to the present invention can be used as a toothpaste for humans and animals and as an ingredient of a mouthwash in addition to a pharmaceutical composition or a food composition, and further, can be used as a feed for animals such as companion animals.

### INDUSTRIAL APPLICABILITY

The composition according to the present invention has industrial applicability as a therapeutic agent, etc.

1. A pharmaceutical composition for preventing and treating periodontal diseases, comprising an Alnus japonica plant extract as an active ingredient.

2. The pharmaceutical composition for preventing and treating periodontal diseases according to claim 1, wherein the Alnus japonica plant extract comprises any one of the following compounds.

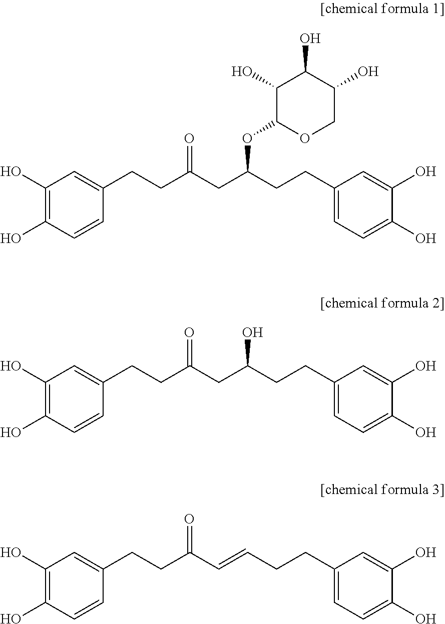

3. The pharmaceutical composition for preventing and treating periodontal diseases according to claim 2, wherein the Chemical Formulas 2 and 2 are obtained by enzymatic digestion of the Alnus japonica plant extract.

4. The pharmaceutical composition for preventing and treating periodontal diseases of claim 3, wherein the pharmaceutical composition for preventing and treating periodontal diseases further comprises an elm extract.

5. A food composition for preventing and alleviating periodontal diseases, comprising an extract of a plant of genus Ulmus as an active ingredient.

6. The food composition for preventing and alleviating periodontal diseases periodontal diseases according to claim 5, wherein the Alnus japonica plant extract comprises any one of the following compounds.

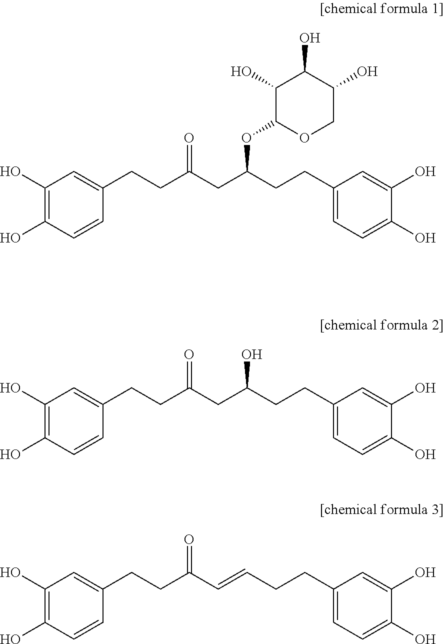

7. The food composition for preventing and alleviating periodontal diseases periodontal diseases according to claim 6, wherein the Chemical Formulas 2 and 3 are obtained by enzymatic digestion of the Alnus japonica plant extract.

8. The food composition for preventing and alleviating periodontal diseases periodontal diseases of claim 7, wherein the food composition for preventing and alleviating periodontal diseases periodontal diseases further comprises an elm extract.

9. A toothpaste composition comprising a plant extract of the genus Ulmus as an active ingredient.

10. The toothpaste or mouthwash composition of claim 9, wherein the Alnus japonica plant extracts include any one of the following compounds:

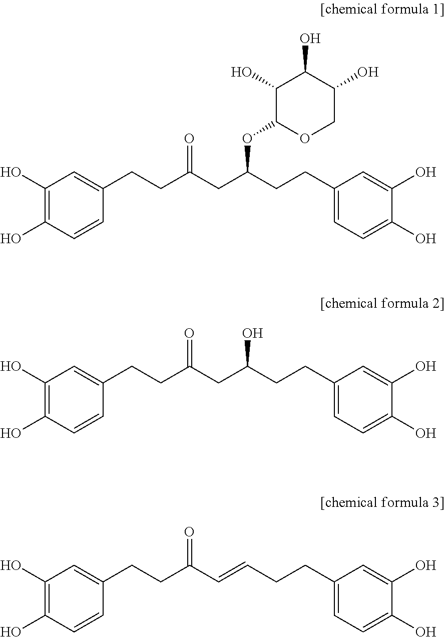

11. The toothpaste or mouthwash composition of claim 10, wherein the Alnus japonica plant extracts are enzymatically decomposed to obtain the Alnus japonica plant extracts.

12. The toothpaste or mouthwash composition of claim 11, wherein the pharmaceutical composition for preventing and treating periodontal diseases further comprises elm extract.

13. A feed comprising a plant extract of the genus Ulmus as an active ingredient.

14. The feed according to claim 13, wherein the Alnus japonica plant extracts comprise any one of the following compounds:

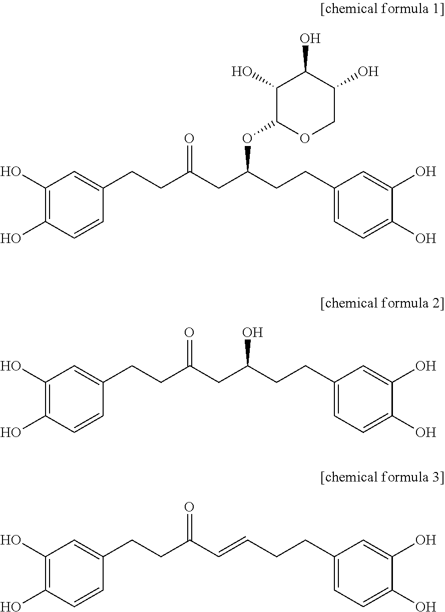

15. The feed according to claim 14, wherein Formula 2 and 3 are obtained by enzymatic digestion of the Alnus japonica plant extracts.

16. The feed according to any one of claim 15, wherein the pharmaceutical composition for preventing and treating periodontal diseases further comprises elm extract.
